(19) United States
(12) Patent Application Publication
(10) Pub. No.: US 2021/0215715 A1
(43) Pub. Date: Jul. 15, 2021

(54) TEST STRIP WITH STRUCTURED SUBSTRATE FOR MULTIPLEX LATERAL FLOW ASSAY FOR DISEASE DIAGNOSTICS

(71) Applicant: Quidel Corporation, San Diego, CA (US)

(72) Inventors: Samantha Chang, San Diego, CA (US); Irene Sinn Blandy, San Diego, CA (US); Robert Reed, San Diego, CA (US); Cristina Charles, San Diego, CA (US); Lauren Hedges, San Diego, CA (US); Ashlee King, San Diego, CA (US)

(21) Appl. No.: 17/144,985

(22) Filed: Jan. 8, 2021

Related U.S. Application Data
(60) Provisional application No. 62/959,727, filed on Jan. 10, 2020.

(51) Int. Cl. G01N 33/68; G01N 33/577; G01N 33/563; G01N 33/569

(52) U.S. Cl. CPC G01N 33/6893; G01N 33/56911; G01N 33/563; G01N 33/577

(57) ABSTRACT

Methods and kits for diagnosis of confirmatory diagnosis of a tick-borne infection, such as Lyme disease, is provided. The methods and kits include one or more multi-channel lateral flow immunoassay test strips. In an embodiment, the test strip comprises at least five individual, discrete fluid flow channels where each of the at least five channels includes one or more binding members (antigens) for detection of immunoglobulin antibodies against a Borrelia bacterium in a blood sample. The binding members (antigens) are, in one embodiment, selected from a group of immunoglobulin antibodies consisting of p18 (decorin-binding protein), p23, p28, p30, p39, p41 (flagellin), p45, p58, p66, p93, VlSE/C6 and p31. The kits include external controls selected from a container with a positive control, a container with a negative control, and both of a container with a positive control and a container with a negative control.

### CROSS-REFERENCE TO RELATED APPLICATIONS

This application claims the benefit of U.S. Provisional Application No. 62/959,727, filed Jan. 10, 2020, which is incorporated herein by reference in its entirety.

### TECHNICAL FIELD

The subject matter described herein relates to lateral flow immunoassays including multiple flow channels for disease diagnostics. More specifically, embodiments as disclosed herein relate to lateral flow immunoassays for detection of Lyme disease.

### BACKGROUND

Lyme disease is caused by infection with the spirochete Borrelia Burgdorferi and is transmitted by the bite of an infected tick of the Ixodid species. It is a systemic disease with variable manifestations, including dermatologic, rheumatologic, neurologic, and cardiac abnormalities. The varying clinical manifestations, coupled with the difficulty in confirming the diagnosis in many patients, have led to some misconceptions about Lyme disease. In most cases, Lyme disease is easily treated and does not progress to the chronic stage.

Culture of Borrelia Burgdorferi is not practical in the microbiology laboratory because of its special growth requirements and very slow growth. Serologic testing can be useful. However, many patients with early disease are seronegative by immunoassay (up to 40%) at the time they present with Erythema migrans. Serologic diagnosis is often not established until later stages of the disease. Testing for Lyme disease typically follows a two-test protocol: The first step employs a sensitive polyvalent screening test enzyme immunoassay (EIA). In early-suspected disease, tests for both IgM and IgG antibodies are recommended. All specimens found to be positive or equivocal by the EIA screening test(s) should be tested by the Western Blot procedure. If the Western Blot is negative, causes of false positive immunoassays should be considered. These include syphilis, subacute bacterial endocarditis (SBE), peridontitis, HIV infection, and hypergammaglobulinemia.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

In one aspect, a method to confirm a diagnosis of Lyme disease in a person with a positive or indeterminate (equivocal) diagnosis of Lyme disease from a first (tier 1) assay is provided. The method includes depositing a blood sample from the person on a multi-channel lateral flow immunoassay comprising at least five individual, discrete fluid flow channels, where each of the at least five channels comprises one or more binding members (antigens) for detection of IgG antibodies against a Borrelia bacterium (e.g., Borrelia burgdorferi), the binding members (antigens) selected from the group consisting of p18 (decorin-binding protein), p23, p28, p30, p39, p41 (flagellin), p45, p58, p66, p93, VlSE/C6 and p31. In some embodiments, (antigens) for detection of IgG antibodies against a Borrelia bacterium, the binding members (antigens) are selected from the group consisting of p18 (decorin-binding protein), p23, p58, p93, and VlSE/C6. The method also includes reporting a confirmatory diagnosis of Lyme disease when the at least five IgG antibodies are present in the blood sample.

In one aspect, a method to confirm a diagnosis of Lyme disease in a person with a positive or equivocal result from a first (tier 1) assay that detects infection by a bacteria of the genus Borrelia includes providing a multi-channel lateral flow immunoassay comprising at least five individual, discrete fluid flow channels, where each of the at least five channels comprises one or more binding members (antigens) for detection of IgG antibodies against a Borrelia bacterium, the binding members (antigens) selected from the group consisting of p18 (decorin-binding protein), p23, p28, p30, p39, p41 (flagellin), p45, p58, p66, p93, VlSE/C6 and p31. The method also includes instructing to place a blood sample from the person on the multi-channel lateral flow immunoassay, wherein a confirmatory diagnosis of Lyme disease occurs when the multi-channel lateral flow immunoassay indicates that the at least five IgG antibodies are present in the blood sample.

In some embodiments, the multi-channel lateral flow immunoassay comprises 10 individual discrete fluid flow channels, wherein each of the binding members p18, p23, p28, p30, p39, p41, p45, p58, p66, p93 are deposited separately in one of the 10 individual channels, and wherein the binding member VlSE/C6, p31 or both is deposited in one or two of the 10 individual channels.

In other embodiments, the multi-channel lateral flow immunoassay comprises 12 individual discrete fluid flow channels, wherein each of the binding members p18, p23, p28, p30, p39, p41, p45, p58, p66, p93, VlSE/C6 are p31 are deposited separately in one of the 12 individual channels.

In one aspect, a two-step immunoassay method to diagnose Lyme disease includes determining a presence or an absence of IgG antibodies, IgM antibodies, or both IgG and IgM antibodies against a Borrelia bacterium in a blood sample from a subject at risk of or suspected of having infection by the Borrelia bacterium, said determining performed using a lateral flow immunoassay comprising binding antigens for said antibodies. The method also includes if said antibodies are present in the blood sample, conducting a confirmatory test by determining a presence or an absence of IgG antibodies in the blood of the subject on a multi-channel lateral flow immunoassay comprising at least five individual, discrete fluid flow channels where each of the at least five channels comprises one or more binding members (antigens) for detection of IgG antibodies against the Borrelia bacterium, the binding members (antigens) selected from the group consisting of p18 (decorin-binding protein), p23, p28, p30, p39, p41 (flagellin), p45, p58, p66, p93, VlSE/C6 and p31, wherein a diagnosis of Lyme disease occurs when the multi-channel lateral flow immunoassay indicates that the at least five IgG antibodies are present in the blood sample.

In some embodiments, a method for diagnosis of Lyme disease in a human subject with symptoms of Lyme disease, includes providing a first assay comprising antigens for determining presence or absence of IgG antibodies, IgM antibodies, or both IgG and IgM antibodies against a Borrelia bacterium. The method also includes providing a second multi-channel lateral flow immunoassay comprising at least five individual, discrete fluid flow channels where each of the at least five channels comprises one or more binding members (antigens) for detection of IgG antibodies against the Borrelia bacterium, the binding members (antigens) selected from the group consisting of p18 (decorin-binding protein), p23, p28, p30, p39, p41 (flagellin), p45, p58, p66, p93, VlSE/C6 and p31. The method also includes optionally providing a third immunoassay test strip comprising binding members for detection of at least three IgM antibodies against the Borrelia bacterium, the binding members selected from the group consisting of p41, p39 and p23, and instructing to deposit or depositing a blood sample from the subject on the first immunoassay to obtain a first result. The method also includes if the first result indicates presence or possible presence of the Borrelia bacterium, depositing or instructing to deposit a blood sample from the subject on the second multi-channel lateral flow immunoassay, and optionally on the third immunoassay, to confirm presence or absence of the Borrelia bacterium in the blood and Lyme disease in the subject.

In some embodiments, a method for confirmatory diagnosis of Lyme disease in a human subject suspected of infection by a Borrelia bacterium, includes providing a first assay comprising antigens for determining presence or absence of IgG antibodies, IgM antibodies, or both IgG and IgM antibodies against a Borrelia bacterium. The method includes providing a second multi-channel lateral flow immunoassay comprising at least five individual, discrete fluid flow channels where each of the at least five channels comprises one or more binding members (antigens) for detection of IgG antibodies against the Borrelia bacterium, the binding members (antigens) selected from the group consisting of p18 (decorin-binding protein), p23, p28, p30, p39, p41 (flagellin), p45, p58, p66, p93, VlSE/C6 and p31. The method also includes optionally providing a third immunoassay test strip comprising binding members for detection of at least three IgM antibodies against the Borrelia bacterium, the binding members selected from the group consisting of p41, p39 and p23, and instructing to deposit or depositing a blood sample from the subject on the first immunoassay to obtain a first result. The method includes if the first result indicates presence or possible presence of the Borrelia bacterium, depositing or instructing to deposit a blood sample from the subject on the second multi-channel lateral flow immunoassay, and optionally on the third immunoassay, to confirm presence or absence of the Borrelia bacterium in the blood and Lyme disease in the subject. In some embodiments, wherein the confirmatory diagnosis is made in a single visit to a medical provider, and the confirmatory diagnosis is completed in less than 2 hours, such as less than 1 hour.

In some embodiments, a kit for confirmatory diagnosis of Lyme disease includes at least three multi-channel lateral flow immunoassay test strips, each test strip comprising at least five individual, discrete fluid flow channels where each of the at least five channels comprises one or more binding members (antigens) for detection of IgG immunoglobulin antibodies against the Borrelia bacterium in the blood sample, the binding members (antigens) selected from a group of IgG immunoglobulin antibodies consisting of p18 (decorin-binding protein), p23, p28, p30, p39, p41 (flagellin), p45, p58, p66, p93, VlSE/C6 and p31. The kit further includes external controls selected from a container with a positive control, a container with a negative control, and both of a container with a positive control and a container with a negative control. In some embodiments, immunoassay test strips of the current technology may also comprise a procedural control zone.

In some embodiments, a kit for confirmatory diagnosis of Lyme disease includes at least three multi-channel lateral flow immunoassay test strips, each immunoassay test strip comprising at least three individual, discrete fluid flow channels where each of the at least three channels comprises one or more binding members (antigens) for detection of IgM antibodies against the Borrelia bacterium, the binding members selected from the group consisting of p19, p23, p39, p41, C10 and VlSE, such as p41, p39 and p23. The kit also includes external controls selected from a container with a positive control, a container with a negative control, and both of a container with a positive control and a container with a negative control.

In another aspect, a method to diagnosis disease due to a tick-borne infection is provided. The method comprises providing a multi-channel lateral flow immunoassay comprising a plurality of individual, discrete fluid flow channels, where each channel in the plurality comprises one or more binding members (antigens) for detection of IgG antibodies and/or IgM antibodies against a tick-borne infection; instructing to place or depositing a blood sample from a person suspected of having disease due to a tick-borne infection on the multi-channel lateral flow immunoassay, and with an instrument, analyzing the multi-channel lateral flow immunoassay to determine presence or absence of IgG antibodies and/or IgM antibodies for each of the binding members, whereby the analysis provides a diagnosis of disease. In some embodiments, wherein the confirmatory diagnosis is made in a single visit to a medical provider and from a single sample, and the confirmatory diagnosis is completed in less than 2 hours, such as less than 1 hour. For example, in some embodiments, immunoassay test strips of the current technology may provide a confirmatory diagnosis of a disease due to a tick-borne infection within about 15 minutes. In some embodiments, the diagnosis may include an “early stage” disease result associated with IgM response and in other embodiments the diagnosis may include a “late stage” disease result associated with an IgG response. In some embodiments, results are obtained by placing immunoassay test strips into an image capturing device to read and/or interpret the results. In some embodiments, the results are obtained by a two-step/tiered algorithm that is conducted by the image capturing device. For example, the algorithm may perform a first analysis of the immunoassay test strip and then only proceed to a second analysis if results from the first step are positive. Furthermore, the results of the test may only be reported as positive, in some embodiments, if both the first and second step analyses provide positive results. For example, if step 1 is positive, proceed to step 2, and if step 2 is negative report negative result. If step 1 is positive, proceed to step 2 and if step 2 is also positive report as positive result. If step 1 is negative, analysis is stopped, step 2 is not performed, and result is reported as negative. In some embodiments, step 1 may comprise an analysis of IgG antibodies while step 2 comprises analysis of IgM antibodies. In other embodiments, step 1 may comprise an analysis of IgM antibodies while step 2 comprises analysis of IgG antibodies. In still other embodiments, step 1 may comprise analysis based on certain antigens while step 2 is based on other antigens, such as tier 1 and tier 2 antigens.

In still another aspect, a kit for diagnosis of disease due to a tick-borne infection is provided. The kit comprises a multi-channel lateral flow immunoassay comprising a plurality of individual, discrete fluid flow channels, where each channel in the plurality comprises one or more binding members (antigens) for detection of IgG antibodies and/or IgM antibodies against a tick-borne infection, wherein the immunoassay is designed to interact with an instrument for analysis to determine presence or absence of IgG antibodies and/or IgM antibodies for each of the binding members, whereby the analysis provides a diagnosis of disease.

In an embodiment, the immunoassay test strip comprises a plurality of first tier IgG antigens and a plurality of second tier IgG antigens. In another embodiment, the immunoassay test strip comprises a plurality of first tier IgM antigens and a plurality of second tier IgM antigens. In one embodiment, the immunoassay test strip is a single test strip that comprises a plurality of first tier IgG antigens, a plurality of second tier IgG antigens, a plurality of first tier IgM antigens and a plurality of second tier IgM antigens. In another embodiment, the immunoassay test strip is a first immunoassay test strip with a plurality of first tier IgG antigens and a plurality of second tier IgG antigen and a second test strip with a plurality of first tier IgM antigens and a plurality of second tier IgM antigens.

In addition to the exemplary aspects and embodiments described above, further aspects and embodiments will become apparent by reference to the drawings and by study of the following descriptions.

Additional embodiments of the present methods and devices, and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present disclosure are set forth in the following description and claims, particularly when considered in conjunction with the accompanying examples and drawings.

### BRIEF DESCRIPTION OF THE DRAWINGS

FIG. 1 illustrates an architecture for diagnosing a tick-borne infection, such as Lyme disease, according to some embodiments.

FIGS. 2A-2B illustrate an exemplary test strip for a tick-borne infection, according to some embodiments.

FIGS. 3A-3B illustrate an exemplary test strip for a tick-borne infection including test channels for immunoglobulin-G (IgG), according to some embodiments.

FIGS. 4A-4B illustrate an exemplary test strip for a tick-borne infection, such as Lyme disease, including test channels for IgG in a different configuration, according to some embodiments.

FIGS. 5A-5C illustrate a structured substrate including fluid control features in a test strip, according to some embodiments.

FIG. 6 is a flow chart illustrating steps in a method to confirm a diagnosis of a tick-borne infection, such as Lyme disease, in a person with a positive or intermediate (equivocal) diagnosis of a tick-borne infection, such as Lyme disease, from a first (tier 1) assay, according to some embodiments.

FIG. 7 is a flow chart illustrating steps in a method to confirm a diagnosis of a tick-borne infection, such as Lyme disease, in a person with a positive or equivocal result from a first (tier 1) assay that detects infection by a bacteria of the genus Borrelia, according to some embodiments.

FIG. 8 is a flow chart illustrating steps in a two-step immunoassay method to diagnose a tick-borne infection, such as Lyme disease, according to some embodiments.

FIG. 9 is a flow chart illustrating steps in a method for diagnosis of a tick-borne infection, such as Lyme disease, in a human subject with symptoms of infection or disease, according to some embodiments.

FIG. 10 is a flow chart illustrating steps in a method for a confirmatory diagnosis of a tick-borne infection, such as Lyme disease, in a human subject suspected of infection by a Borrelia bacterium, according to some embodiments.

FIG. 11 is a block diagram illustrating an example computer system with which the diagnostic architecture of FIG. 1, and the methods disclosed herein can be implemented, according to some embodiments.

FIGS. 12A-12B illustrate an exemplary test strip for a tick-borne infection, such as Lyme disease, including test channels for IgG and IgM in a different configuration, according to some embodiments.

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, references to a “polymer” includes a single polymer as well as two or more of the same or different polymers; reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, and the like, unless the context of the disclosure indicates otherwise, or is inconsistent with such an interpretation. For example, in a list of numerical values such as “about 49, about 50, about 55,” “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The compositions of the present disclosure can comprise, consist essentially of, or consist of, the components disclosed.

All percentages, parts, and ratios are based upon the total weight of the topical compositions and all measurements made are at about 25° C., unless otherwise specified.

The phrase “pharmaceutically acceptable” is employed herein to refer to those compounds, salts, compositions, dosage forms, etc., which are, within the scope of sound medical judgment, suitable for use in contact with the tissues of human beings and/or other mammals without excessive toxicity, irritation, allergic response, or other problems or complications, commensurate with a reasonable benefit/risk ratio. In some aspects, “pharmaceutically acceptable” means approved by a regulatory agency of the federal government or a state government, or listed in the U.S. Pharmacopeia or other generally recognized pharmacopeia for use in mammals (e.g., animals), and more particularly, in humans.

The term “treating” is used herein, for instance, in reference to methods of treating an infection due to an infectious agents, such as Lyme disease infection due to infection by a Borrelia bacterium, and generally includes the administration of a compound or composition, which reduces the frequency of, or delays the onset of, symptoms of the infection in a subject relative to a subject not receiving the compound or composition. This can include reversing, reducing, or arresting the symptoms, clinical signs, and underlying pathology of a condition in a manner to improve or stabilize a subject's condition.

The terms “inhibiting” or “reducing” are used in reference to methods to inhibit or to reduce the extent of infection or symptoms of infection in a population as compared to an untreated control population.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications, and publications are referenced. The disclosures of these patents, patent applications, and publications in their entireties are incorporated into this disclosure by reference, in order to more fully describe the state of the art as known to those skilled therein as of the date of this disclosure. This disclosure will govern in the instance that there is any inconsistency between the patents, patent applications, and publications cited and this disclosure.

For convenience, certain terms employed in the specification, examples, and claims are collected here. Unless defined otherwise, all technical and scientific terms used in this disclosure have the same meanings as commonly understood by one of ordinary skill in the art to which this disclosure belongs.

### II. Methods for Diagnosis

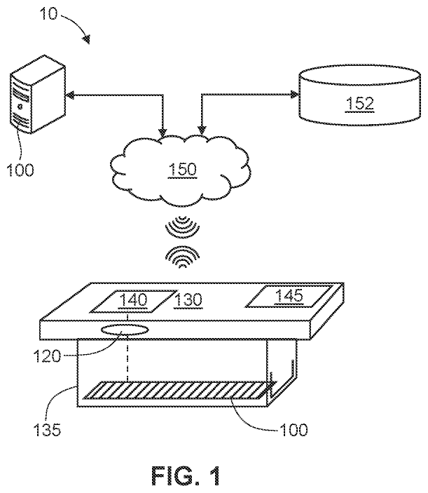

FIG. 1 illustrates an architecture 10 for diagnosing a tick-borne infection, such as Lyme disease, including a remote server 110, a database 152, and an image-capturing device 130 to collect an image from a test strip 100 in enclosure 135, according to some embodiments. In architecture 10, test strip 100 and enclosure 135 may be consumables that the user may dispose of after use. For example, test strip 100 may be replaced after each use of a test sample, while enclosure 135 may be used a few more times. In that regard, test strip 100 and the enclosure may be part of a package requested by the user to a clinical service provider. The package may include one enclosure 135 and multiple test strips 100 that may be used with it. In some embodiments, enclosure 135 may be a semi-permanent or permanent auxiliary box that can be used multiple times (e.g., a cassette or cartridge), independently of whether they are part of the package. In some embodiments, enclosure 135 is a housing or cartridge to ease the handling of test strip 100. In other embodiments, test strip 100 is an immunoassay test strip, such as a dip stick. That is, enclosure 135 is optional, and if present, can be a flexible laminate, such as that disclosed in U.S. Patent Application Publication No. 2009/02263854 and shown in Design Patent No. D606664.

In some embodiments, test strip 100 may be part of a test kit that further includes a device for collecting a blood sample. In some embodiments, the device for collecting a blood sample is configured to separate plasma from the blood sample. In some embodiments, the device for collecting a blood sample includes one or more of a capillary tube, a lancet, a reagent tube, a reagent solution. An exemplary device for collecting and/or dispensing a blood sample is described in U.S. Patent Application Publication No. 2018/0136194, which is incorporated by reference herein. In one embodiment, the device is configured to collect a blood sample and to dispense a plasma sample.

In some embodiments, a test strip of the present technology is configured so that a biological sample, such as a blood sample, can be added directly to a sample introduction port of the test strip without prior processing. In other embodiments, a biological sample, such as a blood sample, may be collected with a collection tool and then added directly a sample introduction port of a test strip without additional processing.

In addition to the consumables, image-capturing device 130 may include a smartphone or other mobile computing device (e.g., tablet, pad, or even laptop) provided by the user. Image-capturing device 130 may generally include a sensor array 140 and an optics coupling mechanism 120 (e.g., a lens system with autofocus capabilities). Image-capturing device 130 may also be configured to couple wirelessly, through a network 150, with a remote server 110 and a remote database 152. Remote server 110 may provide support for an image-capturing application 145 installed in image-capturing device 130. The support may include installation, update, and maintenance of image-capturing application 145, retrieval of raw data (e.g., pictures, sequences of pictures and videos) for storage in database 152, image processing, and the like. In some embodiments, image-capturing device 130 may be configured to collect images from more than one test strips 100, simultaneously, or within the same enclosure 135. Accordingly, in some embodiments, optics coupling mechanism 120 may be configured to capture images from two or more test strips 100, and either interleave image collection rounds, or take images sequentially between the different test strips. For example, in some embodiments, a set of two, three, or more test strips 100 configured for a full Lyme disease diagnostic (e.g., for a single patient) may be tested within the same enclosure 135, using the same image-capturing device 130 (e.g., alternating between multiple frames from each test strip every second or so). To achieve this, optics coupling mechanism 120 may include any one of a lens, a mirror, a planar waveguide, a diffraction grating, a switchable component, or a combination thereof.

While some of the descriptions herein are focused on fluorescence spectroscopic analysis of the test strip, some embodiments consistent with the present disclosure may include any other type of electromagnetic interaction and spectroscopic analysis. Some examples of spectroscopic analysis consistent with the present disclosure may include Raman spectroscopy, infrared absorption spectroscopy, infrared reflectance/transmittance spectroscopy, and the like. Furthermore, in some embodiments, the light emitting source may be replaced by an optical coupling mechanism (e.g., a lens, mirror, prism, diffraction grating or any combination thereof) to use solar radiation (e.g., during day light) or any exterior illumination to excite a spectroscopic response of the area of interest in the test strip.

Enclosure 135 is configured to avoid or control any external light to interfere with the fluorescence excitation light or with the fluorescence emission light collected by the image-capturing device. For example, it is desirable to illuminate the area of interest in the test strip uniformly (e.g., no shadows, bright spots, or other artifacts) to create a smooth spectroscopic background that can be filtered out by the image-capturing application in the image-capturing device.

Some embodiments extract a value for assessing a diagnostic of the assay by spatially and/or spectrally filtering an image of test strip 100. Accordingly, filtered pixel values may be aggregated and compared to a pre-selected threshold. Thus, when the aggregated value is lower or greater than the threshold, a disease diagnosis may be positive. Some embodiments may include error values based on statistical analysis and calibration, to provide a confidence interval for the diagnostics. In other embodiments, the information can be compared between the area that one analyte band takes with a similar area where no capture of the fluorescent complex exists.

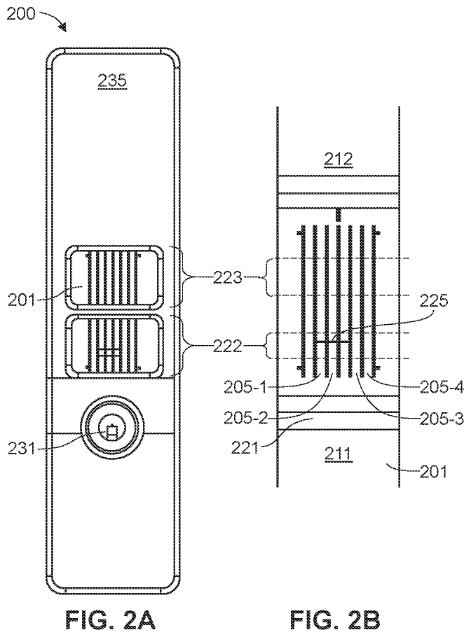

FIGS. 2A-2B illustrates an exemplary test assay 200, according to some embodiments. The test assay comprises a test strip comprising a substrate 201, optionally encased in a housing or cassette 235, which has one or more openings for viewing regions of the test strip. For example, cassette 235 has two openings to permit viewing or interrogation by an instrument of a first capture zone 222 and, optionally, of a second capture or control zone 223. Cassette 235 will also typically include a sample introduction port to receive a patient sample, such as a blood or plasma sample. The sample introduction port, in some embodiments, may include a blood separator 231, which is configured to receive a blood sample from a patient, and separate the serum or plasma from blood cells in the blood sample. In some embodiments, the blood serum or blood plasma is the sample that is placed on the sample introduction port for contact with the test strip, and more particularly, for contact a sample pad 211 in or on substrate 201. An absorbent pad 212 (FIG. 2B) may also be included on the test strip.

In some embodiments, the blood, plasma, or serum sample from the patient may be mixed with a standard solution to form a fluid sample that is received by the sample pad. The standard solution may include control markers not correlated with the tick-borne disease, and which may be used as a control measurement (e.g., negative control and/or positive control) to calibrate the test response. The kits described herein, in some embodiments, are provided with a positive control swab or sample. In some embodiments, a negative control swab or sample is provided. In some embodiments, an external positive control reagent and/or an external negative control reagent is provided in the kit with the assay. A user may be prompted by an instrument that reads the assay to insert or apply a positive or negative control sample or swab into the instrument prior to or after the instrument receives and reads the test assay with the patient sample. In some embodiments, the external control provided in the kit is one or both of a container with a positive control and/or a container with a negative control. In some embodiments, the container with a positive control includes a container with plasma or serum positive for the Borrelia bacterium. In some embodiments, wherein positive control comprises a container with plasma or serum positive for Borrelia burgdorferi. For example, in some embodiments, the plasma or serum in the container with a positive control is IgM positive for Borrelia burgdorferi. In some embodiments, wherein the container with a negative control includes a container with plasma or serum negative for the Borrelia bacterium.

With reference to FIG. 2B, sample pad 211 receives the sample suspected of containing the analyte(s) of interest (e.g., an antibody against a Borrelia bacterium). It will be appreciated that the test assay for detection of antibodies against a Borrelia bacterium, for diagnosis of Lyme disease, is configured for the detection of a plurality of analytes of interest. Reference herein to a single analyte of interest intends reference to multiple analytes of interest where scientifically sensible. After a sample is placed on the sample pad (or in a port that is in communication with a sample pad) on the test strip, the sample flows into conjugate zone 221 which comprises a mobilizable, detectable species that binds the analyte(s) of interest, e.g., an antibody against the infectious agent. The mobilizable, detectable species in the exemplary test strip for detecting infection by a species in the Borrelia genus is, in one embodiment, a non-human, anti-human antibody that has or is associated with a detectable label. The non-human, anti-human antibody is, in some embodiments, a non-human, anti-human IgG antibody bearing a detectable label, such as a fluorescent, chemiluminescent, or other optically detectable tag, such as a bead or chemical moiety.

In one embodiment, optically detectable intends optically detectable by an instrument and not visually detectable by an unaided human eye. In one embodiment, the detectable label is a particle containing a label element, such as a fluorescent element. An exemplary fluorescent element is a lanthanide material, such as one of the fifteen elements lanthanum, cerium, praseodymium, neodymium, promethium, samarium, europium, gadolinium, terbium, dysprosium, holmium, erbium, ytterbium, lutetium, and yttrium. In one embodiment, the lanthanide material is embedded in or on a particle, such as a polystyrene particle. The particles can be microparticles (particles less than about 1,000 micrometers in diameter, in some instances less than about 500 micrometers in diameter, in some instances less than 200, 150, or 100 micrometers in diameter) containing a luminescent or fluorescent lanthanide, wherein in some embodiments, the lanthanide is europium. In some embodiments, the lanthanide is a chelated europium. The particles, in some embodiments, have a core of a lanthanide material with a polymeric coating, such as a europium core with polystyrene coating. A binding partner for the analyte(s) of interest in the sample is/are attached to or associated with the outer surface of the particles.

As mentioned above, the binding partner for the analyte(s) of interest is typically an antibody, a monoclonal antibody, or a polyclonal antibody. One example is a non-human, anti-human IgG antibody. Non-human, anti-human IgG antibodies are exemplified as goat, anti-human antibodies, however the non-human portion of the antibody can be any mammal, including but not limited to mouse, rabbit, rat, sheep, etc. A skilled artisan will appreciate that other binding partners can be selected, and can include complexes such as a biotin and streptavidin complex.

With continued reference to FIGS. 2A-2B, upon entering conjugate zone 221, the liquid sample hydrates, suspends, and/or mobilizes the mobilizable, detectable species. Complexes of the mobilizable, detectable species and the analyte(s) of interest form, assuming the analyte(s) of interest are present. The fluid with the mobilizable, detectable species and complexes continues to move downstream on the test strip to test line(s) or test spot(s), and any control or reference lines if included, disposed on the immunoassay test strip. It will be appreciated that reference to a test “line” or control “line” is a generic reference to a region or position and does not intend a geometric shape of a line, as the test line may in fact be a single dot or an array of dots.

In the embodiment of FIG. 2B, downstream of the conjugate zone 221, the test strip is configured to define a plurality of discrete, individual fluid channels, each channel having a test line with an immobilized specific binding member for binding with the analyte of interest, and capture of the complex of the mobilizable, detectable species and the analyte of interest. Representative individual, discrete fluid flow channels are identified in FIG. 2B as 205-1, 205-2, 205-3, and 205-4. Each channel has a test line, such as the test line identified as 225 in channel 205-2. Test line 225 is, as seen, formed from a drop or from two or more drops of reagent that comprises a specific binding member for the analyte of interest, and may be in the form of a dried line or dot that comprises the specific binding member and any desired excipients.

At the test line, a detectable signal is emitted from the complex of the mobilizable, detectable species and the analyte of interest bound to the specific binding member deposited at the test line. The presence of an emission upon its interrogation by a stimulus from the instrument will be attributable to the presence of the target analyte (e.g., presence of pathogenic antigens, and the like). However, the amount of signal within the boundaries of the immunoassay band may include some background. To better assess the background signal (e.g., not originated by target analytes bound to the antibodies on the band), some test strips may include a control area or zone.

As mentioned above, substrate 201 includes a plurality of fluid channels, and channels 205-1, 205-2, 205-3, and 205-4 are representative (collectively referred to as “fluid channels 205”). Each fluid channel comprises a test line comprising a specific binding member for an analyte to be detected in the patient sample. For detection of tick-borne disease associated with a Borrelia bacterium, antigens with binding affinity for IgG and/or IgM antibodies against the Borrelia bacterium are deposited in each fluid channel at the test line. Accordingly, in some embodiments, each individual discrete fluid channel in the multi-channel immunoassay test strip comprise a binding member (antigen) for detection of IgM (or IgG) antibodies against the Borrelia bacterium. For example, in some embodiments, (antigens) for detection of IgG antibodies against a Borrelia bacterium, the binding members (antigens) are selected from the group consisting of p18 (decorin-binding protein), p23, p58, p93, and VlSE/C6. For example, in some other embodiments, (antigens) for detection of IgM antibodies against a Borrelia bacterium, the binding members (antigens) are selected from the group consisting of p19, p23, p39, p41, C10 and VSE. For example, in some embodiments, the binding members (antigens) for detection of IgM antibodies can comprise the antigens p41, p39 and p23, and the like. More specifically, the fluid channels each comprise a binding antigen such as the 41 kiloDalton (kDa) peptide (p41) that binds an IgM antibody, a 39 kDa peptide (p39) that binds an IgM antibody, and a 23 kDa peptide (p23) that binds an IgM antibody.

In some embodiments, substrate 201 may include one or more fluid channels with no binding member or with a binding member not associated with the disease to be diagnosed to function as a control channel (e.g., a positive control or a negative control, or one or more channels for each). For example, in some embodiments, a control antibody may be included in a standard solution provided with the kit. Accordingly, a measurement protocol may include mixing the standard solution with the patient's blood to prepare the sample fluid before placing in sample pad 211.

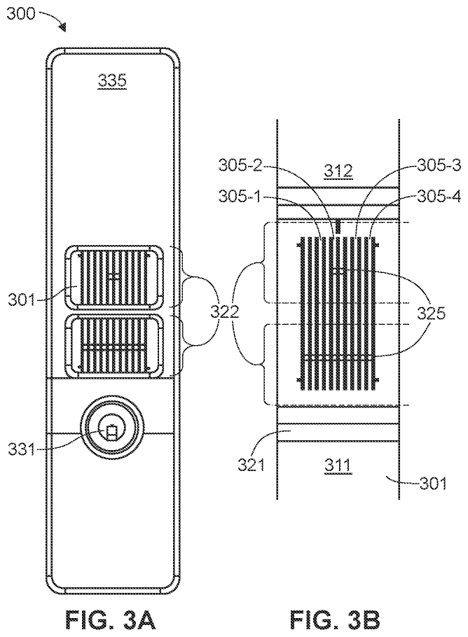

FIGS. 3A-3B illustrate another exemplary test strip 300 for detecting immunoglobulin-G (IgG). A cassette 335, a sample port 331, a sample pad 311, a conjugate zone 321, and an absorbent pad 312 may be as described above In this embodiment, substrate 301 includes ten discrete, individual fluid flow channels, such as 305-1, 305-2, 305-3 and 305-4 (only four marked in the figure, for clarity). At least some of the plurality of fluid channels include reagents 325 at a test line in each channel, that define a capture zone 322. Each reagent is a binding member, such as an antigen peptide, associated an infectious agent and with binding affinity for an IgG antibody (e.g., for the detection of Lyme disease). Accordingly, in some embodiments, each reagent drop in each fluid flow channel may include one binding member (antigens) for detection of IgG antibodies against the Borrelia bacterium. More specifically, each fluid flow channel may comprise a binding member associated with a second tier Western Blot test for IgG in Lyme disease. For example, in some embodiments, binding members (antigens) such as p18 (decorin-binding protein), p23, p28, p30, p39, p41 (flagellin), p45, p58, p66, p93, VlSE/C6 and p31, and the like are on the test strip. In some embodiments, one or more channels may include one or more drops 325 separated by a pre-determined distance along the fluid channel, to accommodate for a larger number of analytes.

In one embodiments, a test strip comprising multiple, discrete and individual fluid channels is contemplated. The test strip is designed to ascertain presence or absence of IgG antibodies and/or IgM antibodies against a Borrelia bacterium.

In one embodiment, a first test strip with multiple, discrete and individual fluid channels is provided, where each channel includes one or more binding members (antigens) for detection of IgG antibodies against a Borrelia bacterium (or other tick-borne infectious agent). In one embodiment, the test strip comprises at least 5, 6, 7, 8, 9, 10, 11, 12, 13, 14, 15, 16, 17, 18, 19 or 20 discrete and individual fluid channels, where each channel comprises a binding member. In one embodiment, the test strip comprises at least 5 discrete and individual fluid channels, where each channel has a single binding member selected from the group consisting of p18 (decorin-binding protein), p23, p28, p30, p39, p41 (flagellin), p45, p58, p66, p93, VlSE/C6 and p31.

In another embodiment, a second test strip with multiple, discrete and individual fluid channels is provided, where each channel includes one or more binding members (antigens) 325 for detection of IgM antibodies against a Borrelia bacterium (or other tick-borne infectious agent). In one embodiment, the test strip comprises at least 5, 6, 7, 8, 9, 10, 11, 12, 13, 14, 15, 16, 17, 18, 19 or 20 discrete and individual fluid channels, where each channel comprises a binding member. In one embodiment, the test strip comprises at least 3 discrete and individual fluid channels, where each channel has a single binding member selected from the group consisting of p23, p39, p41 (flagellin).

In another embodiment, a test strip comprising multiple, discrete and individual fluid channels is provided, where each channel includes one or more binding members (antigens) for detection of IgG and IgM antibodies against a Borrelia bacterium (or other tick-borne infectious agent). The test strip comprises at least 5, 6, 7, 8, 9, 10, 11, 12, 13, 14, 15, 16, 17, 18, 19 or 20 discrete and individual fluid channels, where each channel comprises a binding member. In one embodiment, the test strip comprises at least 5 discrete and individual fluid channels with a binding member for detection of IgG antibodies and at least 2 discrete and individual fluid channels with a binding member for detection of IgM antibodies. The binding members can be any of those described herein.

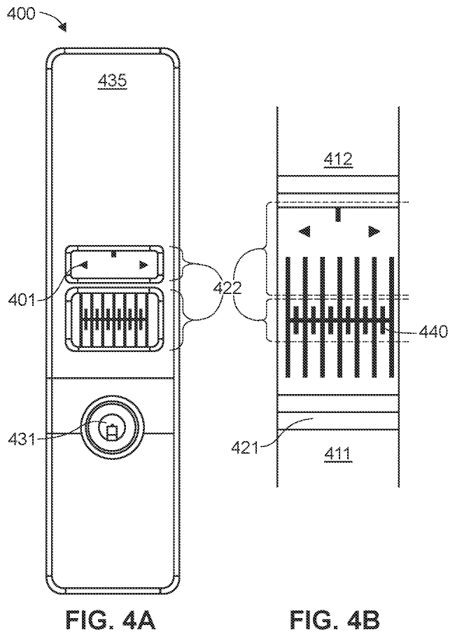

FIGS. 4A-4B illustrate an exemplary test strip 400 with multiple, individual and discrete fluid flow channels. A cassette 435, a sample port 431, a sample pad 411, a conjugate zone 421, and an absorbent pad 412 may be as described above. In this embodiment substrate 401 includes n fluid channels that are further divided into n×2 fluid channels. In this embodiment, n is 6, however it will be appreciated that n can be any integer between 1-100, 1-50, 1-25, 1-20, 1-15, 2-100, 2-50, 2-25, 2-20, 2-15, 2-10, 3-100, 3-50, 3-25, 3-15, or 3-10. Each initial channel n is formed to have a barrier or separating member, such as that identified as member 440, in one of the channels. Fluid initially entering the channel is separated into two fluid streams when it contacts member 440. On either side of member 440 is a test line comprising a binding antigen for the complex that is formed as the sample flows across the conjugate zone 421, as described above. The binding antigens at each test line form a capture zone 422 that is viewable by an instrument to ascertain whether signal is emitted from each discrete test line. In this exemplary test strip, the test lines comprise binding members for detection of IgG and IgM antibodies, using any combinations of the antigens described herein.

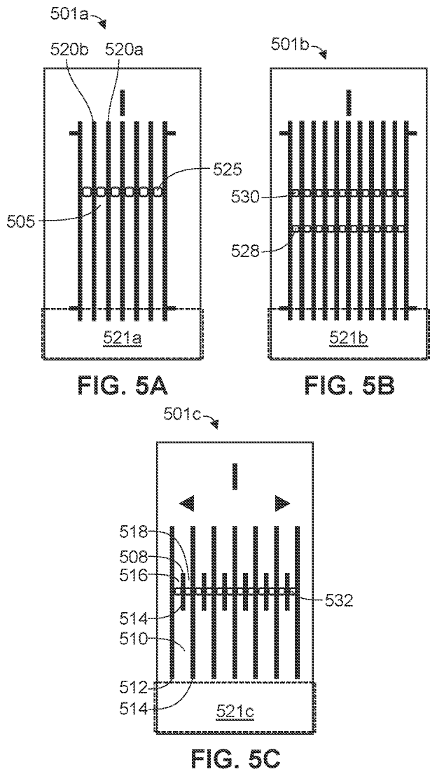

FIGS. 5A-5C illustrate test strips for the immunoassay devices described herein. The test strips comprise a substrate, typically a nitrocellulose substrate, that is treated to define a plurality of fluid flow channels. For example, substrate 501a, 501b, and 501c (hereinafter, collectively referred to as “substrates 501”) in, respectively, FIGS. 5A-5C, are treated to remove substrate in certain regions. By way of example, substrate is removed as indicated by 520a and 520b in FIG. 5A to define between the regions of removed substrate a fluid channel 505. The test strip in FIG. 5A comprises six fluid flow channels, channel 505 being exemplary. The test strip in FIG. 5B comprises 12 fluid flow channels, each channel defined by opposing regions of treated substrate. The test strip in FIG. 5C comprises 6 fluid flow channels, each channel defined by opposing regions of treated substrate, where each of the 6 fluid flow channels has a fluid dividing feature, such as fluid dividing feature 508 in fluid flow channel 510. Fluid flow channel 510 is defined by opposing regions 512, 514, of treated substrate. Fluid dividing feature 508 serves to divide fluid channel 510 into two smaller, parallel channels 516, 518 on either side of fluid dividing feature 508. Thus, the test strip of FIG. 5C has six fluid flow channels that receive fluid directly from a sample zone and/or a conjugate zone (such as conjugate zone 521c), where the six channels are divided downstream by a fluid dividing feature in each channel, to form twelve fluid flow channels.

In an embodiment, the regions of treated substrate, such as 520a, 520b, are treated by chemical or laser etching to remove all or a portion of substrate in the treated region. This is described, for example, in US 2018/0229232, incorporated by reference herein. In one embodiment, the test strip is a laminate of a substrate that is treated to remove all or a portion of substrate in a defined pattern to control fluid flow and a hydrophilic or hydrophobic support layer. The test strips will comprise a conjugate zone and test lines (or test dots) downstream of the conjugate zone. For example, the test strip of FIG. 5A comprises 6 fluid channels, a conjugate zone 521a, and a test dot or in each fluid channel, such as test line 525. The test strip of FIG. 5B comprises 10 fluid channels, each fluid channel comprising two test or capture spots, such as test spots 528, 530. In some embodiments, the two test spots in a single fluid flow channel are comprised of the same reagents and in other embodiments are comprised of different reagents. When comprised of different reagents, the test spots can be for the detection of different analytes of interest. The test spots may be separated along the fluid channel by a pre-selected distance, to avoid crosstalk and/or interference between different signals. In some embodiments, two drops sharing a single fluid channel may conjugate to a different emitter or label element (e.g., a fluorophore for a different wavelength), to avoid or mitigate crosstalk and interference. IN the test strip of FIG. 5C each of the 12 fluid channels comprises a capture line, such as line 532, for binding to an analyte of interest.

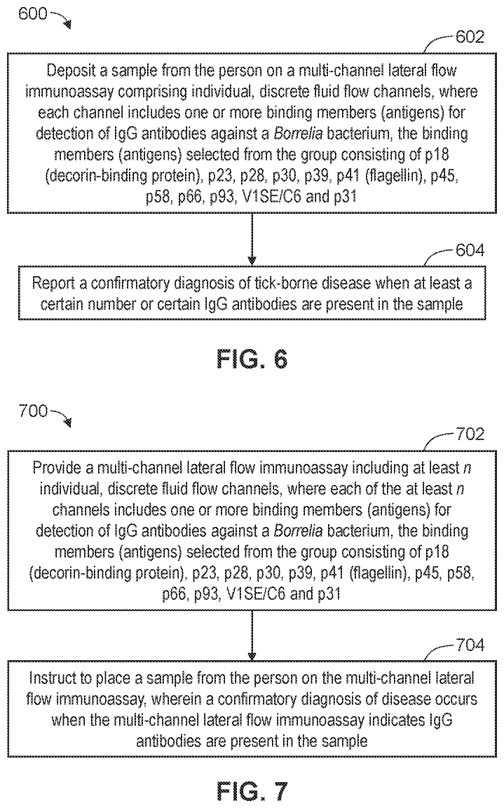

FIG. 6 is a flow chart illustrating steps in a method 600 to confirm a diagnosis of a tick-borne disease (such as Lyme disease) in a person with a positive or intermediate (equivocal) diagnosis of tick-borne disease from a first (tier 1) assay, according to some embodiments. Method 600 may be performed at least partially by a computer or an image-capturing device as in the architecture illustrated in FIG. 1. Accordingly, at least some of the steps in method 600 may be performed by a processor executing instructions stored in a memory. Further, methods consistent with the present disclosure may include at least one step as described in method 600. In some embodiments, methods consistent with the present disclosure include one or more steps in method 600 performed in a different order, simultaneously, almost simultaneously, or overlapping in time.

Step 602 includes depositing a blood, serum or plasma sample from the person on a multi-channel lateral flow immunoassay comprising individual, discrete fluid flow channels, where each of the channels comprises one or more binding members (antigens) for detection of IgG antibodies against a Borrelia bacterium, the binding members (antigens) selected from the group consisting of p18 (decorin-binding protein), p23, p28, p30, p39, p41 (flagellin), p45, p58, p66, p93, VlSE/C6 and p31.

Step 604 includes reporting a confirmatory diagnosis of tick-borne disease when the a certain or a certain number of IgG antibodies are present in the sample.

FIG. 7 is a flow chart illustrating steps in a method 700 to confirm a diagnosis of disease due to a tick-borne infectious agent, such as Lyme disease, in a person with a positive or equivocal result from a first (tier 1) assay that detects infection by a bacteria of the genus Borrelia, according to some embodiments. Method 700 may be performed at least partially by a computer or an image-capturing device as in the architecture illustrated in FIG. 1. Accordingly, at least some of the steps in method 700 may be performed by a processor executing instructions stored in a memory. Further, methods consistent with the present disclosure may include at least one step as described in method 700. In some embodiments, methods consistent with the present disclosure include one or more steps in method 700 performed in a different order, simultaneously, almost simultaneously, or overlapping in time.

Step 702 includes providing a multi-channel lateral flow immunoassay comprising at least n individual, discrete fluid flow channels, where each of the at least n channels comprises one or more binding members (antigens) for detection of IgG antibodies against a Borrelia bacterium, the binding members (antigens) selected from the group consisting of p18 (decorin-binding protein), p23, p28, p30, p39, p41 (flagellin), p45, p58, p66, p93, VlSE/C6 and p31. In various embodiments, n is an integer between 2-50, 2-40, 2-30, 2-25, 2-20, 2-15, 2-12 or 2-10.

Step 704 includes instructing to place a blood, plasma, or serum sample from the person on the multi-channel lateral flow immunoassay. In some embodiments, a confirmatory diagnosis of disease occurs when the multi-channel lateral flow immunoassay indicates that the at least certain or at least a certain number of IgG antibodies are present in the sample.

In some embodiments, the confirmatory diagnosis of disease as in methods 600 or 700 is from a blood sample from a person with symptoms of disease for greater than 30 days. In other embodiments, the confirmatory diagnosis of disease as in methods 600 or 700 is from a blood sample from a person with symptoms of disease or infection for less than 30 days. In some embodiments, methods 600 or 700 may further include as step for depositing a blood, serum or plasma sample from the person on a second lateral flow immunoassay comprising binding members for detection of at least n IgM antibodies against a Borrelia bacterium, the binding members selected from the group consisting of p41, p39 and p23, n is an integer between 1-5. In some embodiments, methods 600 or 700 may further include a step of providing a second lateral flow immunoassay comprising binding members for detection of at least n IgM antibodies against a Borrelia bacterium, the binding members selected from the group consisting of p41, p39 and p23, n is an integer between 1-5, and a step of instructing to place a sample from the person on the second lateral flow immunoassay, wherein a confirmatory diagnosis of disease occurs when the second lateral flow immunoassay indicates that certain or a certain number of IgM antibodies are present in the sample.

In some embodiments, the methods described herein comprise detecting a complex comprised of one of the binding members (antigens), an antibody in the blood sample, and a reporter comprised of an antibody conjugated to a detectable label.

In some embodiments, the second lateral flow immunoassay is a multi-channel lateral flow immunoassay comprising at least three individual, discrete fluid flow channels, wherein each of the three channels comprises one binding member selected from p41, p39 and p23.

In some embodiments, any one of methods 600 or 700 may include the steps of detecting a complex comprised of one of the binding members (antigens), an antibody in the sample, and a reporter comprised of an antibody conjugated to a detectable label. In some embodiments, the detectable label is a fluorescent particle. In some embodiments, the reporter is deposited on and mobilizable on the lateral flow immunoassay. In some embodiments, methods 600 or 700 may further include the steps of treating, administering, and/or recommending treatment with a therapeutic agent, such as an antibiotic. In some embodiments, the first (tier 1) assay is selected from the group consisting of an enzyme immunoassay, an indirect immunofluorescence, and a lateral flow immunoassay. In some embodiments, the first (tier 1) assay is an in vitro, qualitative assay for detection of human IgM antibody to individual proteins of a Borrelia bacterium in human, blood, serum or plasma in a person. In some embodiments, the first (tier 1) assay is an enzyme immunoassay or an immunofluorescence assay. In some embodiments, the immunofluorescence assay is a lateral flow immunoassay. In some embodiments, depositing a blood sample or instructing to place a blood sample in any one of methods 600 or 700 includes depositing a serum or plasma sample or instructing to place a serum or plasma sample.

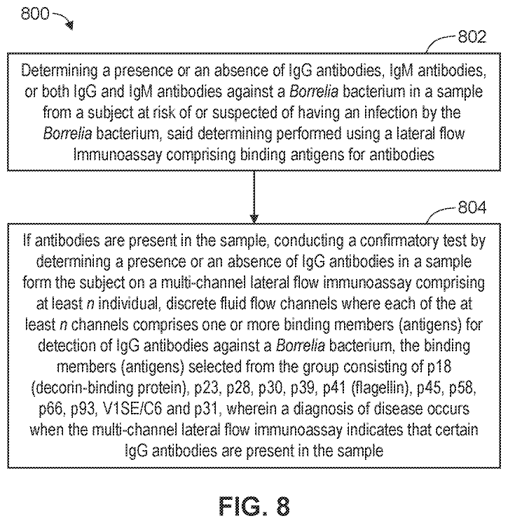

FIG. 8 is a flow chart illustrating steps in a two-step immunoassay method 800 to diagnose a tick-borne disease, such as Lyme disease, according to some embodiments. Method 800 may be performed at least partially by a computer or an image-capturing device as in the architecture illustrated in FIG. 1. Accordingly, at least some of the steps in method 800 may be performed by a processor executing instructions stored in a memory. Further, methods consistent with the present disclosure may include at least one step as described in method 800. In some embodiments, methods consistent with the present disclosure include one or more steps in method 800 performed in a different order, simultaneously, almost simultaneously, or overlapping in time.

Step 802 includes determining a presence or an absence of IgG antibodies, IgM antibodies, or both IgG and IgM antibodies against a Borrelia bacterium in a blood, plasma or serum sample from a subject at risk of or suspected of having infection by the Borrelia bacterium, the determining performed using a lateral flow immunoassay comprising binding antigens for the antibodies.

Step 804 includes conducting a confirmatory test when the antibodies are present in the sample, by determining a presence or an absence of IgG antibodies in the blood, plasma or serum of the subject on a multi-channel lateral flow immunoassay comprising at least n individual, discrete fluid flow channels where each of the at least n channels comprises one or more binding members (antigens) for detection of IgG antibodies against a Borrelia bacterium, the binding members (antigens) selected from the group consisting of p18 (decorin-binding protein), p23, p28, p30, p39, p41 (flagellin), p45, p58, p66, p93, VlSE/C6 and p31, wherein a diagnosis of disease occurs when the multi-channel lateral flow immunoassay indicates that certain or a certain number, (e.g., at least 3, 4, 5, 6, 7, 8,) IgG antibodies are present in the sample. In various embodiments, n is an integer between 2-50, 2-40, 2-30, 2-25, 2-20, 2-15, 2-12 or 2-10.

It will be appreciated that the step 804 of conducting a confirmatory test may be conducting additional analysis of a test strip or a data gathered from a test strip, as described elsewhere herein.

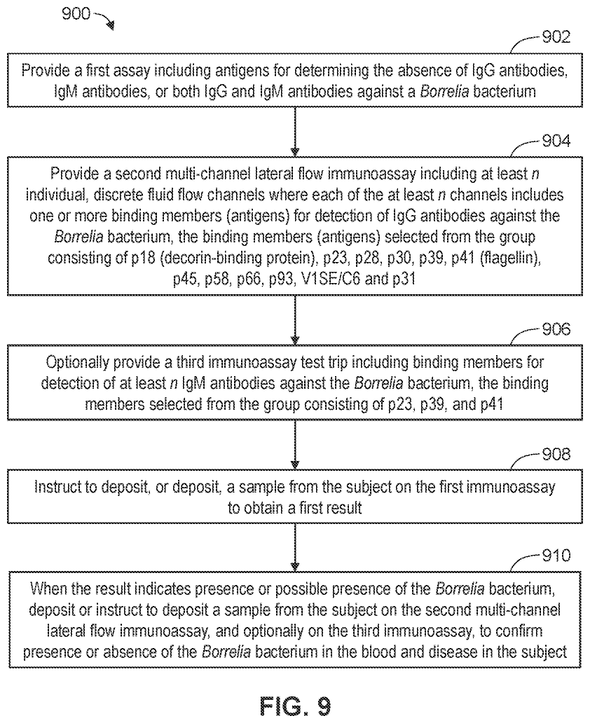

FIG. 9 is a flow chart illustrating steps in a method 900 for diagnosis of a disease due to an infectious agent transmitted by a tick, such as Lyme disease, in a human subject with symptoms of the disease of concern, according to some embodiments. Method 900 may be performed at least partially by a computer or an image-capturing device as in the architecture illustrated in FIG. 1. Accordingly, at least some of the steps in method 900 may be performed by a processor executing instructions stored in a memory. Further, methods consistent with the present disclosure may include at least one step as described in method 900. In some embodiments, methods consistent with the present disclosure include one or more steps in method 900 performed in a different order, simultaneously, almost simultaneously, or overlapping in time.

Step 902 includes providing a first assay comprising antigens for determining presence or absence of IgG antibodies, IgM antibodies, or both IgG and IgM antibodies against a Borrelia bacterium. Step 904 includes providing a second multi-channel lateral flow immunoassay comprising at least n individual, discrete fluid flow channels where each of the at least n channels comprises one or more binding members (antigens) for detection of IgG antibodies against the Borrelia bacterium, the binding members (antigens) selected from the group consisting of p18 (decorin-binding protein), p23, p28, p30, p39, p41 (flagellin), p45, p58, p66, p93, VlSE/C6 and p31. In various embodiments, n is an integer between 2-50, 2-40, 2-30, 2-25, 2-20, 2-15, 2-12 or 2-10. In some embodiments, n is 2, 3, 4, 5, 6, 7, 8, 9, or 10.

Step 906 includes optionally providing a third immunoassay test strip comprising binding members for detection of at least n IgM antibodies against the Borrelia bacterium, the binding members selected from the group including p23, p39, and p41. In some embodiments, n is 2, 3, 4, 5, 6, 7, 8, 9, or 10.

Step 908 includes instructing to deposit or depositing a blood, serum or plasma sample from the subject on the first immunoassay to obtain a first result.

Step 910 includes depositing or instructing to deposit a blood, serum or plasma sample from the subject on the second multi-channel lateral flow immunoassay when the first result indicates presence or possible presence of the Borrelia bacterium, and optionally on the third immunoassay, to confirm presence or absence of the Borrelia bacterium in the blood and disease in the subject.

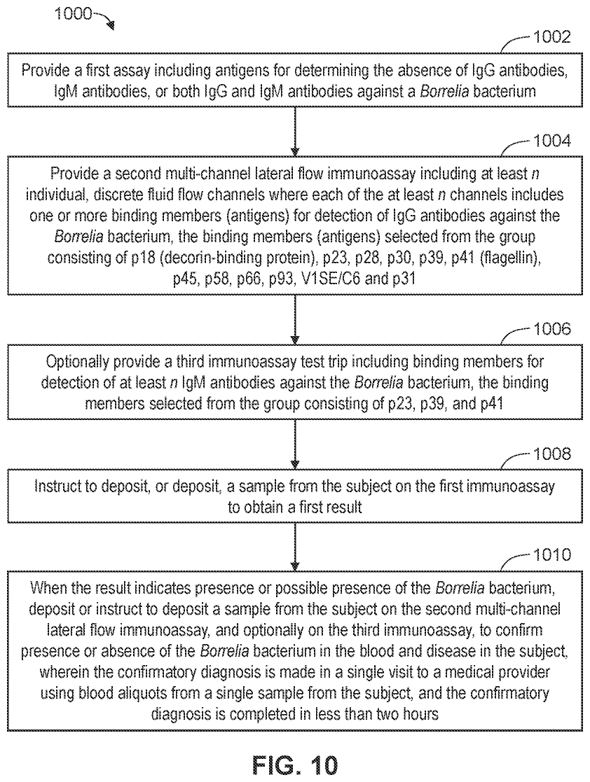

FIG. 10 is a flow chart illustrating steps in a method 1000 for a confirmatory diagnosis of Lyme disease in a human subject suspected of infection by a Borrelia bacterium, according to some embodiments. Method 1000 may be performed at least partially by a computer or an image-capturing device as in the architecture illustrated in FIG. 1. Accordingly, at least some of the steps in method 1000 may be performed by a processor executing instructions stored in a memory. Further, methods consistent with the present disclosure may include at least one step as described in method 1000. In some embodiments, methods consistent with the present disclosure include one or more steps in method 1000 performed in a different order, simultaneously, almost simultaneously, or overlapping in time.

Step 1002 includes providing a first assay comprising antigens for determining presence or absence of IgG antibodies, IgM antibodies, or both IgG and IgM antibodies against a Borrelia bacterium. Step 1004 includes providing a second multi-channel lateral flow immunoassay comprising at least n individual, discrete fluid flow channels where each of the at least n channels comprises one or more binding members (antigens) for detection of IgG antibodies against the Borrelia bacterium, the binding members (antigens) selected from the group consisting of p18 (decorin-binding protein), p23, p28, p30, p39, p41 (flagellin), p45, p58, p66, p93, VlSE/C6 and p31. In some embodiments, n is 2, 3, 4, 5, 6, 7, 8, 9, 10, 11, 12, 13, or 14.

Step 1006 includes optionally providing a third immunoassay test strip comprising binding members for detection of at least three IgM antibodies against the Borrelia bacterium, the binding members selected from the group including p23, p39, and p41.

Step 1008 includes instructing to deposit or depositing a sample from the subject on the first immunoassay to obtain a first result. Step 1010 includes if the first result indicates presence or possible presence of the Borrelia bacterium, depositing or instructing to deposit a sample from the subject on the second multi-channel lateral flow immunoassay, and optionally on the third immunoassay, to confirm presence or absence of the Borrelia bacterium in the blood and disease in the subject.

In some embodiments, the confirmatory diagnosis is made in a single visit to a medical provider using blood aliquots from a single blood sample from the subject, and the confirmatory diagnosis in step 1010 is completed in less than two hours.

In some embodiments, the confirmatory diagnosis in steps 1002-1010 is made in a single visit to a medical provider using aliquots from a single sample from the subject. In some embodiments, a single collection may provide enough sample from a patient to run both IgM/IgG assays. In yet other embodiments, a sample for IgM and a separate sample for IgG may be desirable, to have enough signal in the assay. For example, in some embodiments, a physician may desire to collect three (3) 25 μl finger stick blood samples from a single finger prick if there is enough sample present.

In some embodiments, at least one or more of steps 1002-1010 may be completed in less than 2 hours. In some embodiments, steps 1002-1010 may be completed within one hour, or even less (e.g., 20 minutes or so).

For example, in some embodiments, steps 1002-1010 may be performed for both IgM and IgG tests within 30 (=2×15) minutes. Using a single image-capturing device, the tests included in steps 1002 and 1010 would take approximately 10-45 minutes to complete a two-tiered (e.g., Lyme) disease test, depending on the antibody levels. Having more than one image-capturing device, or multiplexing one image-capturing device to perform more than one lateral assay simultaneously, or almost simultaneously, reduces the time to completion proportionately.

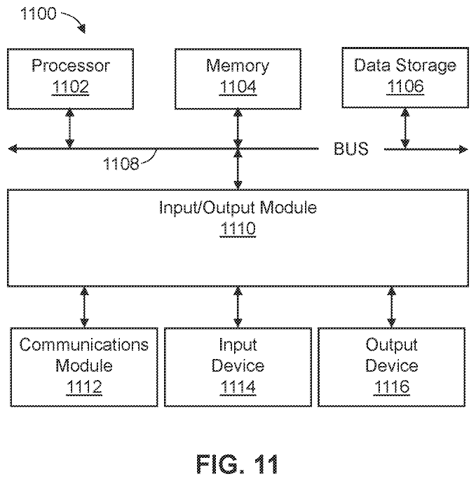

FIG. 11 is a block diagram illustrating an example computer system 1100 with which the image-capturing device and the server of FIG. 1, and the methods disclosed herein (e.g., methods 600-1000, cf. FIGS. 6-10), can be implemented, according to some embodiments. In certain aspects, computer system 1100 may be implemented using hardware or a combination of software and hardware, either in a dedicated server, or integrated into another entity, or distributed across multiple entities.

Computer system 1100 (e.g., server 110, image-capturing device 130) includes a bus 1108 or other communication mechanism for communicating information, and a processor 1102 coupled with bus 1108 for processing information. By way of example, computer system 1100 may be implemented with one or more processors. Processor 14102 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information.

Computer system 1100 can include, in addition to hardware, code that creates an execution environment for the computer program in question, e.g., code that constitutes processor firmware, a protocol stack, a database management system, an operating system, or a combination of one or more of them stored in an included memory 1104, such as a Random Access Memory (RAM), a flash memory, a Read-Only Memory (ROM), a Programmable Read-Only Memory (PROM), an Erasable PROM (EPROM), registers, a hard disk, a removable disk, a CD-ROM, a DVD, or any other suitable storage device, coupled to the bus for storing information and instructions to be executed by processor 1402. Processor 1102 and memory 1104 can be supplemented by, or incorporated in, special purpose logic circuitry.

The instructions may be stored in memory 1104 and implemented in one or more computer program products, i.e., one or more modules of computer program instructions encoded on a computer-readable medium for execution by, or to control the operation of, computer system 1100, and according to any method well-known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command-line interface languages, compiled languages, concurrent languages, curly-bracket languages, dataflow languages, data-structured languages, declarative languages, esoteric languages, extension languages, fourth-generation languages, functional languages, interactive-mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and xml-based languages. The memory may also be used for storing temporary variable or other intermediate information during execution of instructions to be executed by the processor.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors 1102 executing one or more computer programs to perform functions by operating on input data and generating output.

Computer system 1100 further includes a data storage device 1106 such as a magnetic disk or optical disk, coupled to the bus for storing information and instructions. Computer system 1100 may be coupled via an input/output module 1410 to various devices. Input/output module 1110 can be any input/output module. Exemplary input/output modules include data ports such as USB ports. Input/output module 1110 may be configured to connect to a communications module. Exemplary communications modules include networking interface cards, such as Ethernet cards and modems. In certain aspects, input/output module 1110 may be configured to connect to a plurality of devices, such as a communications module 1112, an input device 1114, and/or an output device 1116. Exemplary input devices 1114 include a keyboard and a pointing device, e.g., a mouse or a trackball, by which a user can provide input to the computer system. Other kinds of input devices 1114 can be used to provide for interaction with a user as well, such as a tactile input device, visual input device, audio input device, or brain-computer interface device. For example, feedback provided to the user can be any form of sensory feedback, e.g., visual feedback, auditory feedback, or tactile feedback; and input from the user can be received in any form, including acoustic, speech, tactile, or brain wave input. Exemplary output devices 1116 include display devices, such as an LCD (liquid crystal display) monitor for displaying information to the user.

In some embodiments, computer system 1100 is a network-based, voice-activated device accessed by the user. Input/output devices 1114 and 1116 may include a microphone providing the queries in voice format, and receiving multiple inputs from the user also in a voice format, in the language of the user. Further, in some embodiments, a neural linguistic algorithm may cause the voice-activated device to contact the user back and receive a user selection of the respiratory mask via a voice command or request.

According to one aspect of the present disclosure, image-capturing device 130 and server 110 can be implemented using computer system 1100 in response to processor 1102 executing one or more sequences of one or more instructions contained in memory 1104. Such instructions may be read into memory 1104 from another machine-readable medium, such as data storage device 1106. Execution of the sequences of instructions contained in the main memory causes processor 1102 to perform the process steps described herein. One or more processors in a multi-processing arrangement may also be employed to execute the sequences of instructions contained in the memory. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system 1100 that includes a back-end component, e.g., as a data server, or that includes a middleware component, e.g., an application server, or that includes a front-end component, e.g., image-capturing device 130 having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. The communication network (e.g., network 150) can include, for example, any one or more of a LAN, a WAN, the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

Computer system 1100 can include image-capturing devices and servers wherein the image-capturing device and server are generally remote from each other and typically interact through a communication network (e.g., image-capturing device 130, server 110, and network 150, cf. FIG. 1). The relationship of image-capturing device and server arises by virtue of computer programs running on the respective computers and having an image-capturing device-server relationship to each other. The computer system can be, for example, and without limitation, a desktop computer, laptop computer, or tablet computer. The computer system can also be embedded in another device, for example, and without limitation, a mobile telephone, a PDA, a mobile audio player, a Global Positioning System (GPS) receiver, a video game console, and/or a television set top box.

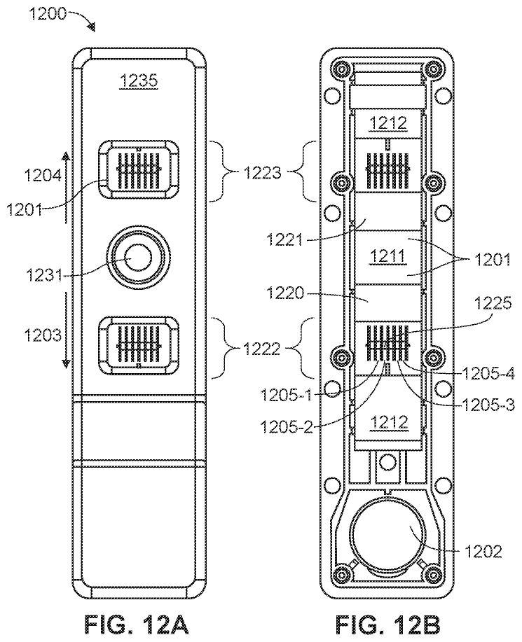

FIGS. 12A-12B illustrate an exemplary test assay 1200, according to some embodiments. The test assay comprises a test strip comprising a substrate 1201, optionally encased in a housing or cassette 1235, which has one or more openings for viewing regions of the test strip. For example, cassette 1235 has two openings to permit viewing or interrogation by an instrument of a first capture zone 1222 and, optionally, of a second capture or control zone 1223.

In some embodiments, cassette 1235 includes a central sample introduction port 1231 to receive a patient sample, such as a blood or plasma sample, and distribute the sample into bidirectional fluid flow paths 1203 and 1204. In some embodiments, the central sample introduction port 1231 is positioned to distribute the sample along a first fluid flow path 1203 to a first plurality of individual, discrete fluid flow channels of a first capture zone 1222 and along a second fluid flow path 1204 to a second a plurality of individual, discrete fluid flow channels of a second capture zone 1223. In some embodiments, the first plurality of individual, discrete fluid flow channels comprises one or more binding members (antigens) for detection of IgG antibodies against a tick-borne infection and the second plurality of individual, discrete fluid flow channels comprises one or more binding members (antigens) for detection of IgM antibodies against a tick-borne infection. In some embodiments, the first plurality of individual, discrete fluid flow channels comprises one or more binding members (antigens) for detection of IgM antibodies against a tick-borne infection and the second plurality of individual, discrete fluid flow channels comprises one or more binding members (antigens) for detection of IgG antibodies against a tick-borne infection. In some embodiments, the one or more binding members (antigens) for detection of IgG comprises a plurality of first tier IgG antigens and a plurality of second tier IgG antigens. In some embodiments, the one or more binding members (antigens) for detection of IgM comprises a plurality of first tier IgM antigens and a plurality of second tier IgM antigens.

The central sample introduction port 1231, in some embodiments, may include a blood separator, which is configured to receive a blood sample from a patient, and separate the serum or plasma from blood cells in the blood sample. In some embodiments, the blood serum or blood plasma is the sample that is placed on the sample introduction port for contact with the test strip, and more particularly, for contact with sample pad 1211 in or on substrate 1201. An absorbent pad 1212 (FIG. 12B) may also be included on the test strip at either end of the bidirectional flow paths 1203 and 1204. In some embodiments, cassette 1235 also includes a desiccant 1202.

In some embodiments, first capture zone 1222 may comprise antigens for detection of IgG antibodies and second capture zone 1223 may comprise antigens for detection of IgM antibodies. In other embodiments, first capture zone 1222 may comprise antigens for detection of IgM antibodies and second capture zone 1223 may comprise antigens for detection of IgG antibodies.

In some embodiments, first capture zone 1222 may comprise antigens for detection of IgG antibodies against a Borrelia bacterium, such as antigens selected from the group consisting of p18 (decorin-binding protein), p23, p58, p93, and VlSE/C6. In some other embodiments, first capture zone 1222 may comprise antigens for detection of IgM antibodies against a Borrelia bacterium, such as antigens selected from the group consisting of p19, p23, p39, p41, C10 and VSE.

In some embodiments, second capture zone 1223 may comprise antigens for detection of IgG antibodies against a Borrelia bacterium, such as antigens selected from the group consisting of p18 (decorin-binding protein), p23, p58, p93, and VlSE/C6. In some other embodiments, second capture zone 1223 may comprise antigens for detection of IgM antibodies against a Borrelia bacterium, such as antigens selected from the group consisting of p19, p23, p39, p41, C10 and VSE.

With further reference to FIG. 12B, sample pad 1211 receives the sample suspected of containing the analyte(s) of interest (e.g., an antibody against a Borrelia bacterium). It will be appreciated that the test assay for detection of antibodies against a Borrelia bacterium, for diagnosis of Lyme disease, is configured for the detection of a plurality of analytes of interest. Reference herein to a single analyte of interest intends reference to multiple analytes of interest where scientifically sensible. After a sample is placed on the sample pad (or in a port, such as central sample introduction port 1231, that is in communication with a sample pad) on the test strip, the sample flows into conjugate zones 1220 and 1221 which comprise, in some embodiments, a mobilizable, detectable species that binds the analyte(s) of interest, e.g., an antibody against the infectious agent. The mobilizable, detectable species in the exemplary test strip for detecting infection by a species in the Borrelia genus is, in one embodiment, a non-human, anti-human antibody that has or is associated with a detectable label. The non-human, anti-human antibody is, in some embodiments, a non-human, anti-human IgG or IgM antibody bearing a detectable label, such as a fluorescent, chemiluminescent, or other optically detectable tag, such as a bead or chemical moiety.

As mentioned above, the binding partner for the analyte(s) of interest is typically an antibody, a monoclonal antibody, or a polyclonal antibody. One example is a non-human, anti-human IgG or IgM antibody. Non-human, anti-human IgG or IgM antibodies are exemplified as goat, anti-human antibodies, however the non-human portion of the antibody can be any mammal, including but not limited to mouse, rabbit, rat, sheep, etc. A skilled artisan will appreciate that other binding partners can be selected, and can include complexes such as a biotin and streptavidin complex.

With continued reference to FIGS. 12A-12B, upon entering conjugate zones 1220 and 1221, the liquid sample hydrates, suspends, and/or mobilizes the mobilizable, detectable species. Complexes of the mobilizable, detectable species and the analyte(s) of interest form, assuming the analyte(s) of interest are present. The fluid with the mobilizable, detectable species and complexes continues to move downstream on the test strip to test line(s) or test spot(s) present in capture zones 1222 and 1223, and any control or reference lines if included, disposed on the immunoassay test strip. It will be appreciated that reference to a test “line” or control “line” is a generic reference to a region or position and does not intend a geometric shape of a line, as the test line may in fact be a single dot or an array of dots.

In the embodiment of FIG. 12B, downstream of the conjugate zones 1220 and 1221, the test strip is configured to define a plurality of discrete, individual fluid channels, each channel having a test line with an immobilized specific binding member for binding with the analyte of interest, and capture of the complex of the mobilizable, detectable species and the analyte of interest. Representative individual, discrete fluid flow channels are identified in FIG. 12B as 1205-1, 1205-2, 1205-3, and 1205-4. Each channel has a test line, such as the test line identified as 1225 in channel 1205-2. Such discrete fluid flow channels are present in both capture zones 1222 and 1223. Test line 1225 is, as seen, formed from a drop or from two or more drops of reagent that comprises a specific binding member for the analyte of interest, and may be in the form of a dried line or dot that comprises the specific binding member and any desired excipients.

At the test lines, such as 1225, in capture zones 1222 and 1223, a detectable signal is emitted from the complex of the mobilizable, detectable species and the analyte of interest bound to the specific binding member deposited at the test line. The presence of an emission upon its interrogation by a stimulus from the instrument will be attributable to the presence of the target analyte (e.g., presence of pathogenic antigens, and the like). However, the amount of signal within the boundaries of the immunoassay band may include some background. To better assess the background signal (e.g., not originated by target analytes bound to the antibodies on the band), some test strips may include a control area or zone.

As mentioned above, substrate 1201 includes a plurality of fluid channels, and channels 1205-1, 1205-2, 1205-3, and 1205-4 are representative (collectively referred to as “fluid channels 1205”) fluid channels of capture zone 1222 as shown. Similar fluid flow channels are also present and shown but not numbered in capture zone 1223. Each fluid channel comprises a test line comprising a specific binding member for an analyte to be detected in the patient sample. For detection of tick-borne disease associated with a Borrelia bacterium, antigens with binding affinity for IgG and/or IgM antibodies against the Borrelia bacterium are deposited in each fluid channel at the test line. Accordingly, in some embodiments, each individual discrete fluid channel in the multi-channel immunoassay test strip comprise a binding member (antigen) for detection of IgM (or IgG) antibodies against the Borrelia bacterium. For example, in some embodiments, (antigens) for detection of IgG antibodies against a Borrelia bacterium, the binding members (antigens) are selected from the group consisting of p18 (decorin-binding protein), p23, p58, p93, and VlSE/C6. For example, in some other embodiments, (antigens) for detection of IgM antibodies against a Borrelia bacterium, the binding members (antigens) are selected from the group consisting of p19, p23, p39, p41, C10 and VSE. For example, in some embodiments, the binding members (antigens) for detection of IgM antibodies can comprise the antigens p41, p39 and p23, and the like. More specifically, the fluid channels each comprise a binding antigen such as the 41 kiloDalton (kDa) peptide (p41) that binds an IgM antibody, a 39 kDa peptide (p39) that binds an IgM antibody, and a 23 kDa peptide (p23) that binds an IgM antibody.

In some embodiments, substrate 1201 may include one or more fluid channels with no binding member or with a binding member not associated with the disease to be diagnosed to function as a control channel (e.g., a positive control or a negative control, or one or more channels for each). For example, in some embodiments, a control antibody may be included in a standard solution provided with the kit. Accordingly, a measurement protocol may include mixing the standard solution with the patient's blood to prepare the sample fluid before placing in sample pad 1211.

A method and kit for diagnosis of disease due to a tick-borne infection is also contemplated. In this method, a kit with one or two immunoassay test strips provides for a complete diagnosis, as will now be described. Diagnosis of tick-borne infections, such as Lyme disease, is presently a two-tiered process, with a first tier to ‘rule in’ infection by the bacteria and a second tier to rule out disease. That is, a first assay (Tier I) is conducted and if it provides a positive or equivocal result, a second test is conducted. The first test is generally a immunoassay, such as an enzyme immunoassay or an immunofluorescence assay. The second test (Tier II), often a Western blot, is one to detect IgM and IgG antibodies if the signs or symptoms in the patient have been present for 30 days or less or is one to detect IgG antibodies if the signs or symptoms in the patient have been present for greater than 30 days. Contemplated herein is a method to conduct both the Tier I and Tier II assays to diagnose disease due to a tick-borne infection.

Two embodiments of the method are contemplated. In a first embodiment, two immunoassay test strips are provided. In a second embodiment, a single immunoassay test strip is provided. Both embodiments are now described. In some embodiments, confirmatory diagnosis of Lyme disease is performed by methods and kits provided herein wherein the assay for diagnosis excludes a Western Blot test or other immunoblot test.

In the first embodiment, a first immunoassay test strip detects IgG immunoglobulins (antibodies) and a second immunoassay test strip detects IgM immunoglobulins (antibodies). The two test strips may be provided in a single kit, along with a positive control, a negative control and instructions for use. The IgG immunoassay test strip comprises a plurality of first tier IgG antigens and a plurality of second tier IgG antigens. The IgM immunoassay test strip comprises a plurality of first tier IgM antigens and a plurality of second tier IgM antigens. With the IgG immunoassay test strip, a complete IgG analysis (1^{st} tier and 2^{nd} tier) on a single test strip is or may be performed, and with the IgM immunoassay test strip, a complete IgM analysis (1^{st} tier and 2^{nd} tier) on a single test strip is or may be performed. Together, the analysis from each of the IgG immunoassay test strip and the IgM immunoassay test strip provide for a diagnosis of disease, with no need for a second confirmatory diagnosis.

In the second embodiment, a single immunoassay test strip comprises a plurality of first tier IgG antigens, a plurality of second tier IgG antigens, a plurality of first tier IgM antigens and a plurality of second tier IgM antigens. The single immunoassay test strip is designed to conduct a complete IgM/IgG diagnosis (1^{st} tier and 2^{nd} tier) on a single test cassette. Thereby, the patient can receive a complete diagnosis (IgM, IgG, or IgM+IgG) from a single blood sample placed on a single test strip in a single visit to a medical provider.

In both embodiments, the immunoassay test strips are designed to interact with an instrument that reads a signal or detectable label, as described above. The instrument is programmed to evaluate signal associated with the antigens designated as tier 1 antigens using a first tier cut-off and/or algorithm. If the analysis from the signal associated with the tier 1 antigens is positive, the instrument proceeds to evaluate signal associated with the antigens designated as tier 2 antigens using a second tier cut-off and/or algorithm. It will be appreciated that the instrument may also be programmed to conduct an analysis of tier 1 antigens and tier 2 antigens, rather than performing analysis of the antigens in a sequential manner (1st tier followed by 2^{nd} tier).

The tick-borne diseases include, but are not limited to, Lyme disease, anaplasmosis, b. miyamotoi, ehrlichiosis, rocky mountain spotted fever, rickettsia, tick-borne relapsing fever, tularemia, and babesia. By way of example, for a diagnosis of Lyme disease due to a tick-borne infection of a Borrelia bacterium (e.g., B. burgdorferi, B. afzelii, and/or B. garinii), the analysis used in the method, whether a sequential tiered approach or non-sequential, single analysis approach can be: (1) determine the number of test bands or test spots that emit signal indicating presence of an antibody for the antigen (example, 4 of 10 bands for IgG antigens emit signal); (2) analyze the test bands or test spots according to a predetermined algorithm to determine positivity (examples: if the test spot for C6 emits signal (i.e., is positive) OR if a combination of p18, p39, p41 OR p18, p39, p41 emit signal (i.e, are positive) AND the test spot of p45 does not emit signal (i.e., is negative)), (3) apply a weighted algorithm based on the positivity bands (example: if C6 is positive, THEN apply a pre-preprogrammed cutoff to the bands for p18 and p41; and if p18 and p41 is positive, THEN apply another, different pre-preprogrammed cutoff to the bands for p23 and p58, etc.). By way of example, Lyme disease associated with infection from B. burgdorferi sensu stricto, B. burgdorferi sensu lato, Borrelia afzelii, Borrelia garinii, Borrelia valaisiana, Borrelia lusitaniae, Borrelia spielmanii, or Borrelia bissetti are contemplated as tick-borne infections and possible resulting disease detectable using the methods and kits described herein.

With regard to a method for complete Lyme diagnosis, antigens on the immunoassay test strip(s) may include native, recombinant and/or peptide forms of the following proteins from Borrelia burgdorferi sensu lato (s.l): p18, p23, p28, p30, p41, p39, p45, p58, p66, p100, VlsE, p31, NapA, Erp family of lipoproteins, BBA family of proteins, BBK family of proteins, outer surface proteins Osp A-F, OppA family of proteins, HSP90, LA7. These proteins may be derived from any of the strains of B. burgdorferi s.l. and/or be used in the detection of any or all strains of B. burgdorferi s.l.

Additional antigens may be incorporated to aid in the diagnosis of other tick-borne diseases including: Borrelia miyamotoi: GlpQ; Anaplasma phagocytophilum: Msp5, OmpA, p44 (Msp2), Asp 62, Asp 55; Babesia microti: IRA, p32, p41; Bartonella henselae: p17, p26, SucB.

In embodiments, the method contemplates an immunoassay test strip that includes about one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, or between about n-30, n-29, n-28, n-27, n-26, n-25, n-24, n-23, n-22, n-21, n-20, n-19, n-18, n-17, n-16, n-15, n-14, n-13, n-12, n-11, n-10, n-9, n-8, n-7, n-6, n-5, n-4, n-3, n-2, where n is 1, 2, 3, 4, 5, 6, 7, 8, 9, 10, 11, 12, 13, 14, 15, 16, 17, 18, 19, 20, 21, 22, 23, 24, 25, 26, 27, 28 or 29 antigens selected from the antigens in the following table.

| Antigen/protein | Description of Antigen |
|---|---|
| P14 | 14 kDa |
| DbpA/Osp17 | 17-18 kDA, outer surface protein decorin |
| | binding protein A |
| P18 | P18 flagellin fragment |
| outer surface protein E (OspE, p19) | 19.2 kDa, outer surface protein, complement |
| | factor H binding |
| outer surface protein C (OspC) | 21-24 kDa, plasmid-coded lipoprotein |
| Crasp 3 | 21 kDa; complement regulator-acquiring |
| | surface protein |
| P21 | Immunogenic integral membrane lipoprotein |
| P22 | Immunogenic integral membrane lipoprotein |
| pG | 22 kDa |
| BBA 36 | 22 kDa B. bugdorferi antigen |
| OspF (p26) | 26.1 kDa, outer membrane protein |
| P28 | OspD, Oms28 |
| P30 | 30 kDa |
| OspA, outer surface protein A | 31 kDa, outer membrane protein |
| OspB, outer surface protein B | 34 kDa, outer membrane protein |
| fibronectin-binding protein BBK32 | 35 kDa |
| (p35) | |
| VlsE, variable major protein-like | 35 kDa; surface exposed lipoprotein |
| sequence E | |
| flagellar protein (FlaA) | 37-38 kDa |
| flagellar protein (FlaB) | |
| BBK32/p35 fibronectic | Fibronectin-binding protein |
| BBK50/p37 | 37 kDa |
| BmpA (p39) | 39 kDa; Borrelial membrane protein A |
| P41 flagellin | 41 kDa |
| P41int | 14 kDa, internal part of flagellin |
| BBO 323 | 42 kDa |
| P58 | 58 kDa |
| P66 membrane associated porin | 66 kDa |
| P71 | 71 kDa |
| P73 | 73 kDa |
| P83 (p83/100) | 83-100 kDa, high molecular mass protein |
| P93 | Immunodominant protoplasmic cylinder antigen |
| HSP, heat shock protein | 60 kDa |
| decorin-binding protein A (DbpA) | |
| decorin-binding protein B (DbpB) | |
| C6, sixth invariant region epitope | |
| oligopeptide permease A2 (OppA2) | |

In one embodiment, a first immunoassay test strip comprises a plurality of first tier IgG antigens and a plurality of second tier IgG antigens, where the first tier antigens and/or the second tier antigens can be any combination or any number of antigens described herein. An IgM immunoassay test strip comprises a plurality of first tier IgM antigens and a plurality of second tier IgM antigens, where the first tier antigens and/or the second tier antigens can be any combination or any number of antigens described herein. In another embodiment, a single immunoassay test strip comprises a plurality of first tier IgG antigens, a plurality of second tier IgG antigens, a plurality of first tier IgM antigens and a plurality of second tier IgM antigens where the first tier antigens and/or the second tier antigens can be any combination or any number of antigens described herein.

In summary, a method for detection of infection and confirmation of diagnosis of a disease due to infection using one or two test strips and a single blood sample is contemplated. The test strips comprise multiple antigens that are recognized by the instrument in a first tier analysis and multiple antigens that are recognized by the instrument in a second tier analysis. It will be appreciated that some of antigens in the second tier analysis may overlap with antigens in the first tier analysis. The one or two test strips provide(s) sufficient IgG and IgM antigens for a complete analysis of disease status, and the instrument is programmed to conduct analysis of groups of the antigens. The antigens described herein above, and those described in WO2017/035389 which is incorporated by reference herein, can be grouped into any number of combinations for a first tier IgG, second tier IgG, first tier IgM and second tier IgM analysis.

While a number of exemplary aspects and embodiments have been discussed above, those of skill in the art will recognize certain modifications, permutations, additions, and sub-combinations thereof. It is therefore intended that the following appended claims and claims hereafter introduced are interpreted to include all such modifications, permutations, additions, and sub-combinations as are within their true spirit and scope.

It is claimed:

1. A method to diagnosis disease due to a tick-borne infection, comprising:
providing a multi-channel lateral flow immunoassay comprising a plurality of individual, discrete fluid flow channels, where each channel in the plurality comprises one or more binding members (antigens) for detection of IgG antibodies and/or IgM antibodies against a tick-borne infection,
instructing to place or depositing a blood sample from a person suspected of having disease due to a tick-borne infection on the multi-channel lateral flow immunoassay, and
with an instrument, analyzing the multi-channel lateral flow immunoassay to determine presence or absence of IgG antibodies and/or IgM antibodies for each of the binding members, whereby the analysis provides a diagnosis of disease.

2. The method of claim 1, wherein the providing step comprises providing an immunoassay test strip with a plurality of first tier IgG antigens and a plurality of second tier IgG antigens.

3. The method of claim 1, wherein the providing step comprises providing an immunoassay test strip with a plurality of first tier IgM antigens and a plurality of second tier IgM antigens.

4. The method of claim 1, wherein the immunoassay comprises a single test strip.

5. The method of claim 1, wherein the multi-channel lateral flow immunoassay comprises a single test strip comprising a central sample introduction port and bidirectional fluid flow paths.

6. The method of claim 5, wherein the central sample introduction port is positioned to distribute the sample along a first fluid flow path to a first plurality of individual, discrete fluid flow channels and along a second fluid flow path to a second a plurality of individual, discrete fluid flow channels.

7. The method of claim 6, wherein the first plurality of individual, discrete fluid flow channels comprises one or more binding members (antigens) for detection of IgG antibodies against a tick-borne infection and the second plurality of individual, discrete fluid flow channels comprises one or more binding members (antigens) for detection of IgM antibodies against a tick-borne infection.

8. The method of claim 7, wherein the one or more binding members (antigens) for detection of IgG comprises a plurality of first tier IgG antigens and a plurality of second tier IgG antigens.

9. The method of claim 8, wherein the one or more binding members (antigens) for detection of IgG are selected from a group consisting of p18 (decorin-binding protein), p23, p28, p30, p39, p41 (flagellin), p45, p58, p66, p93, VlSE/C6 and p31.

10. The method of claim 7, wherein the one or more binding members (antigens) for detection of IgM comprises a plurality of first tier IgM antigens and a plurality of second tier IgM antigens.

11. The method of claim 10, wherein the one or more binding members (antigens) for detection of IgM are selected from a group consisting of p19, p23, p39, p41, C10 and VlSE.

12. The method of claim 1, wherein the disease due to a tick-borne infection is Lyme disease.

13. The method of claim 1, wherein the blood sample is from a person with symptoms of Lyme disease for less than 30 days.

14. The method of claim 1, wherein the multi-channel lateral flow immunoassay is an in vitro, qualitative assay for detection of human IgM and/or IgG antibodies to individual proteins of a Borrelia bacterium in human, blood, serum or plasma.

15. The method of claim 14, wherein the Borrelia bacterium is Borrelia burgdorferi.

16. A kit for diagnosis of disease due to a tick-borne infection, comprising:
a multi-channel lateral flow immunoassay comprising a plurality of individual, discrete fluid flow channels, where each channel in the plurality comprises one or more binding members (antigens) for detection of IgG antibodies and/or IgM antibodies against a tick-borne infection, wherein the immunoassay is designed to interact with an instrument for analysis to determine presence or absence of IgG antibodies and/or IgM antibodies for each of the binding members, whereby the analysis provides a diagnosis of disease.

17. The kit of claim 16, wherein the multi-channel lateral flow immunoassay comprises a plurality of first tier IgG antigens and a plurality of second tier IgG antigens.

18. The kit of claim 16, wherein the multi-channel lateral flow immunoassay comprises a plurality of first tier IgM antigens and a plurality of second tier IgM antigens.

19. The kit of claim 16, wherein the multi-channel lateral flow immunoassay is a single test strip.

20. The kit of claim 16, wherein the multi-channel lateral flow immunoassay comprises a single test strip comprising a central sample introduction port and bidirectional fluid flow paths.

21. The kit of claim 20, wherein the central sample introduction port is positioned to distribute the sample along a first fluid flow path to a first plurality of individual, discrete fluid flow channels and along a second fluid flow path to a second a plurality of individual, discrete fluid flow channels.

22. The kit of claim 21, wherein the first plurality of individual, discrete fluid flow channels comprises one or more binding members (antigens) for detection of IgG antibodies against a tick-borne infection and the second plurality of individual, discrete fluid flow channels comprises one or more binding members (antigens) for detection of IgM antibodies against a tick-borne infection.

23. The kit of claim 22, wherein the one or more binding members (antigens) for detection of IgG comprises a plurality of first tier IgG antigens and a plurality of second tier IgG antigens.

24. The kit of claim 23, wherein the one or more binding members (antigens) for detection of IgG are selected from a group consisting of p18 (decorin-binding protein), p23, p28, p30, p39, p41 (flagellin), p45, p58, p66, p93, VlSE/C6 and p31.

25. The kit of claim 22, wherein the one or more binding members (antigens) for detection of IgM comprises a plurality of first tier IgM antigens and a plurality of second tier IgM antigens.

26. The kit of claim 25, wherein the one or more binding members (antigens) for detection of IgM are selected from a group consisting of p19, p23, p39, p41, C10 and VlSE.

27. The kit of claim 16, further comprising external controls selected from a container with a positive control, a container with a negative control, and both of a container with a positive control and a container with a negative control.

28. The kit of claim 16, wherein the disease due to a tick-borne infection is Lyme disease.

29. The kit of claim 16, wherein the multi-channel lateral flow immunoassay is an in vitro, qualitative assay for detection of human IgM and/or IgG antibodies to individual proteins of a Borrelia bacterium in human, blood, serum or plasma.

30. The kit of claim 29, wherein the Borrelia bacterium is Borrelia burgdorferi.
